(19) United States
(12) Patent Application Publication
(10) Pub. No.: US 2020/0338124 A1
(43) Pub. Date: Oct. 29, 2020

(54) CELL

(71) Applicant: AUTOLUS LIMITED, London (GB)

(72) Inventors: Shaun Cordoba, London (GB); Evangelia Kokalaki, London (GB); Martin Pulé, London (GB); Simon Thomas, London (GB); Shimobi Onuoha, London (GB)

(21) Appl. No.: 16/613,364

(22) PCT Filed: May 14, 2018
(86) PCT No.: PCT/GB18/51294

§ 371 Date: Nov. 13, 2019

(30) Foreign Application Priority Data
May 15, 2017 (GB) 1707779.3

(51) Int. Cl. A61K 35/17; C07K 16/28; C07K 14/725; C07K 14/705; C12N 15/86

(52) U.S. Cl. CPC A61K 35/17; C07K 16/2803; C07K 16/2896; C07K 14/7051; C07K 2319/33; C07K 14/70596; C07K 14/70517; C12N 15/86; C07K 2319/03; C07K 14/70521

(57) ABSTRACT

The present invention provides a cell which co-expresses a first chimeric antigen receptor (CAR) and second CAR wherein the first CAR comprises an activating endodomain and the second CAR comprises an inhibitory endodomain, wherein the inhibitory endodomain comprises C-terminal Src Kinase (CSK).

### FIELD OF THE INVENTION

The present invention relates to a cell which comprises more than one chimeric antigen receptor (CAR).

### BACKGROUND TO THE INVENTION

A number of immunotherapeutic agents have been described for use in cancer treatment, including therapeutic monoclonal antibodies (mAbs), immunoconjugated mAbs, radioconjugated mAbs and bi-specific T-cell engagers.

Typically these immunotherapeutic agents target a single antigen: for instance, Rituximab targets CD20; Myelotarg targets CD33; and Alemtuzumab targets CD52.

However, it is relatively rare for the presence (or absence) of a single antigen effectively to describe a cancer, which can lead to a lack of specificity. Targeting antigen expression on normal cells leads to on-target, off-tumour toxicity.

Most cancers cannot be differentiated from normal tissues on the basis of a single antigen. Hence, considerable “on-target off-tumour” toxicity occurs whereby normal tissues are damaged by the therapy. For instance, whilst targeting CD20 to treat B-cell lymphomas with Rituximab, the entire normal B-cell compartment is depleted, whilst targeting CD52 to treat chronic lymphocytic leukaemia, the entire lymphoid compartment is depleted, whilst targeting CD33 to treat acute myeloid leukaemia, the entire myeloid compartment is damaged etc.

The predicted problem of “on-target off-tumour” toxicity has been bourne out by clinical trials. For example, an approach targeting ERBB2 caused death to a patient with colon cancer metastatic to the lungs and liver. ERBB2 is over-expressed in colon caner in some patients, but it is also expressed on several normal tissues, including heart and normal vasculature.

Chimeric Antigen Receptors (CARs)

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

However, the use of CAR-expressing T cells is also associated with on-target, off tumour toxicity. For example, a CAR-based approach targeting carboxy anyhydrase-IX (CAIX) to treat renal cell carcinoma resulted in liver toxicity which is thought to be caused by the specific attack on bile duct epithelial cells (Lamers et al (2013) Mol. Ther. 21:904-912.

There is therefore is a need for alternative CAR-based T cell approaches with increased selectivity and with reduced on target, off tumour toxicity.

### DESCRIPTION OF THE FIGURES

FIG. 1: (a) Generalized architecture of a CAR: A binding domain recognizes antigen; the spacer elevates the binding domain from the cell surface; the trans-membrane domain anchors the protein to the membrane and the endodomain transmits signals. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in cis.

FIG. 2: Schematic diagram illustrating CAR Logic gates

CAR T-cell receptors can be engineered to respond to logical rules of target cell antigen expression. This is best illustrated with an imaginary FACS scatter-plot. Target cell populations may express both, either or neither antigens “A” and “B”. Different target populations (marked by a cross) are killed by T-cells transduced with a pair of CARs connected by different gates. In an AND gate, single positive targets are spared, whereas double positive targets are killed (bottom left). With an AND NOT gate, double-positive targets are preserved while single-positive targets “B-expressing” target cells are killed (bottom right).

FIG. 3: Creation of target cell populations

SupT1 cells were used as target cells. These cells were transduced to express either CD19, CD33 or both CD19 and CD33. Target cells were stained with appropriate antibodies and analysed by flow cytometry.

FIG. 4: Cartoon showing a version of the cassette used to generate cells expressing both an activating CAR and an inhibitory CAR with a CSK endodomain

A first and second CAR, comprising activating and inhibiting endodomains respectively, were co-expressed using a foot-and-mouth disease (FMD) 2A peptide sequence. Signal1 is a signal peptide derived from IgG1 (but can be any effective signal peptide). scFv1 is the single-chain variable segment which recognizes CD19 (but can be a scFv or peptide loop or ligand or in fact any domain which recognizes any desired arbitrary target). STK is the human CD8 stalk but may be any non-bulky extracellular domain. CD28tm is the CD28 trans-membrane domain but can by any stable type I protein transmembrane domain and CD3Z is the CD3 Zeta endodomain but can be any endodomain which contains ITAMs. The activatory endodomain of this CAR may further comprise any one or more of OX40, CD28 and/or 4-1BB costimulatory signals (not shown). Signal2 is a signal peptide derived from CD8 but can be any effective signal peptide which is different in DNA sequence from Signal1. scFv2 recognizes CD33 but as for scFv1 is arbitrary. muSTK is the mouse CD8 stalk but can be any spacer which co-localises but does not cross-pair with that of the activating CAR. muCD8tm is the murine CD8a trans-membrane and truncated endodomain but can by any stable type I protein transmembrane domain with a truncated endodomain. tkCSK is the tyrosine kinase domain of C-terminal Src kinase (CSK). This CAR comprising an inhibitory endodomain may comprise full length CSK.

FIG. 5: Amino acid sequence of two CAR constructs comprising (a) tyrosine kinase domain of CSK (tkCSK) or (b) full length CSK (CSK).

FIG. 6: Design rules for building logic gated CAR T-cells.

OR, AND NOT and AND gated CARs are shown in cartoon format with the target cell on top, and the T-cell at the bottom with the synapse in the middle. Target cells express arbitrary target antigens A, and B. T-cells express two CARs which comprise of anti-A and anti-B recognition domains, spacers and endodomains.

An AND NOT gate requires a design which result in co-segregation of both CARs upon recognition of both antigens. For antigens of similar size, or for target epitopes which are a similar distance from the target cell membrane, this may be achieved using similar sized spacers.

An AND gate requires a design which results in kinetic segregation of the two CARs at the T-cell:target cell synapse upon recognition of both antigens. For antigens of similar size, or for target epitopes which are a similar distance from the target cell membrane, this may be achieved by choosing different spacers, one of which is longer/more bulky than the other, as described in WO2015/075469. For target epitopes which are spatially separate in terms of their distance from the target cell membrane, kinetic segregation may be achievable with similar sized spacers, as described in WO 2017/068361.

FIG. 7(a): Cytotoxicity (72 h) of CAR T cell constructs for SupT1 cells. To measure cytotoxic capacity of the CAR constructs were challenged against the SupT1 cell line. 72 hours after the T cells and SupT1 cells were co-cultured, the absolute number of SupT1 target cells was calculated, and the number in the CAR normalised according to the target number in the non-transduced (NT) condition. The normalised data are expressed as a percentage of cell survival. The INO-CSK LT22-H CAR construct having a first CAR comprising an activatory endodomain and a second CAR comprising a CSK inhibitory endodomain shows a higher overall percentage of cell survival compared to the LT22-Hinge CAR construct which lacks a CSK inhibitory endodomain when challenged with non-ligand expressing target cells. The INO-CSK LT22-H CAR reduces non-specific killing.

FIG. 7(b): Cytotoxicity (72 h) of CAR T cell constructs for SupT1 CD22 cells. To measure cytotoxic capacity of the CAR constructs were challenged against the SupT1 CD22 target cell line. 72 hours after the T cells and SupT1 CD22 cells were co-cultured, the absolute number of SupT1 CD22 target cells was calculated, and the number in the CAR normalised according to the target number in the non-transduced (NT) condition. The normalised data are expressed as a percentage of cell survival. The INO-CSK LT22-H CAR construct having a first CAR comprising an activatory endodomain and a second CAR comprising the CSK inhibitory endodomain shows a significantly higher overall percentage of target cell survival compared to the LT22-Hinge CAR construct, which lacks a CSK inhibitory endodomain.

FIG. 8: T-cell proliferation (day 7) histograms when challenged with Raji target cells. CD56-depeleted CAR expressing T cells were analysed by flow cytometry to measure the dilution of the Cell Trace Violet (CTV) which occurs as the T-cells divide. The T cells labelled with CTV are excited with a 405 nm (violet) laser. Proliferation of the CAR construct cells comprising a CSK inhibitory endodomain (INO-CSK LT22-H) is shown to be reduced for the donor tested compared to the construct lacking the inhibitory endodomain (LT22-Hinge).

FIG. 9: IFN-γ cytokine production from CAR T-cells challenged with Raji target cells (72 h). CAR constructs with different endodomains were compared for IFN-γ secretion after 72 h co-culture with Raji target cells. The INO-CSK LT22-H CAR construct comprising a CSK inhibitory endodomain shows less IFN-γ secretion than the LT22-Hinge construct, which lacks the CSK inhibitory endodomain.

### SUMMARY OF ASPECTS OF THE INVENTION

The present inventors have previously developed a panel of “logic-gated” chimeric antigen receptor pairs which, when expressed by a cell, such as a T cell, are capable of detecting a particular pattern of expression of at least two target antigens. If the at least two target antigens are arbitrarily denoted as antigen A and antigen B, the three possible options are as follows:

“OR GATE”—T cell triggers when either antigen A or antigen B is present on the target cell “AND GATE”—T cell triggers only when both antigens A and B are present on the target cell “AND NOT GATE”—T cell triggers if antigen A is present alone on the target cell, but not if both antigens A and B are present on the target cell

Engineered T cells expressing this CAR combination can be tailored to be exquisitely specific for cancer cells, based on their particular expression and lack of expression of two or more markers.

WO2015/075469 and WO2015/075470 describe dual CAR-based T cell approaches with selectivity for expression/non-expression of a pattern of at least two antigens presented on the target cell, in which downstream signalling of TCR is inhibited by coexpression of a phosphatase domain.

The inventors have now surprisingly found that the tyrosine kinase domain of C-terminal Src Kinase (CSK) can be used as an inhibitory endodomain in CAR-based logic gate.

Thus in a first aspect, the present invention provides a cell which co-expresses a first chimeric antigen receptor (CAR) and second CAR wherein the first CAR comprises an activating endodomain and the second CAR comprises an inhibitory endodomain, wherein the inhibitory endodomain comprises a tyrosine kinase domain of C-terminal Src Kinase (CSK).

The cell may be an immune effector cell, such as a T-cell or natural killer (NK) cell. Features mentioned herein in connection with a cell apply equally to other immune effector cells, such as T cells or NK cells.

The first and second CAR of the cell may comprise (i) an antigen binding domain, (ii) a spacer, (iii) a trans-membrane domain, and (iv) an endodomain.

The spacers of the first and second CARs of the cell may be orthologous, such as mouse and human CD8 stalks.

The inhibitory endodomain of the CAR of the cell may comprise the amino acid sequence SEQ ID NO: 15 or SEQ ID NO: 16.

The first CAR of the cell may comprise an antigen-binding domain which binds CD33 and the second CAR of the cell may comprise an antigen-binding domain which binds CD34.

As explained in the introduction, acute myeloid leukaemia (AML) cells express CD33. Normal stem cells express CD33 but also express CD34, while AML cells are typically CD34 negative. Targeting CD33 alone to treat AML is associated with significant toxicity as it depletes normal stem cells. However, specifically targeting cells which are CD33 positive but not CD34 positive avoids this considerable off-target toxicity. Thus in the present invention, the first CAR comprising the activating endodomain may comprise an antigen-binding domain which binds CD33 and the second CAR which comprises the inhibitory endodomain may comprise an antigen-binding domain which binds CD34.

In a second aspect, the present invention provides a nucleic acid construct encoding both the first and second chimeric antigen receptors (CARs) as defined in the first aspect of the invention.

The nucleic acid cosntruct according to the second aspect may have the following structure:AgB1-spacer1-TM1-endo1-coexpr-AgB2-spacer2-TM2-endo2

in which

AgB1 is a nucleic acid sequence encoding the antigen-binding domain of the first CAR;

spacer 1 is a nucleic acid sequence encoding the spacer of the first CAR;

TM1 is a a nucleic acid sequence encoding the transmembrane domain of the first CAR;

endo 1 is a nucleic acid sequence encoding the activating endodomain of the first CAR;

coexpr is a nucleic acid sequence enabling co-expression of both CARs

AgB2 is a nucleic acid sequence encoding the antigen-binding domain of the second CAR;

spacer 2 is a nucleic acid sequence encoding the spacer of the second CAR;

TM2 is a a nucleic acid sequence encoding the transmembrane domain of the second CAR;

endo 2 is a nucleic acid sequence encoding the inhibitory endodomain of the second CAR;

which nucleic acid sequence, when expressed in a cell, encodes a polypeptide which is cleaved at the cleavage site such that the first and second CARs are co-expressed at the cell surface.

The nucleic acid construct allowing co-expression of two CARs may encode a self-cleaving peptide or a sequence which allows alternative means of co-expressing two CARs such as an internal ribosome entry sequence or a 2^{nd} promoter or other such means whereby one skilled in the art can express two proteins from the same vector.

Alternative codons may be used in regions of construct encoding the same or similar amino acid sequences, in order to avoid homologous recombination.

In a third aspect, the present invention provides a kit which comprises
(i) a first nucleic acid sequence encoding the first chimeric antigen receptor (CAR) as defined in the first aspect of the invention, which nucleic acid sequence has the following structure:

AgB1-spacer1-TM1-endo1

in which

AgB1 is a nucleic acid sequence encoding the antigen-binding domain of the first CAR;

spacer 1 is a nucleic acid sequence encoding the spacer of the first CAR;

TM1 is a a nucleic acid sequence encoding the transmembrane domain of the first CAR;

endo 1 is a nucleic acid sequence encoding the endodomain of the first CAR; and
(ii) a second nucleic acid sequence encoding the second chimeric antigen receptor (CAR) as defined in the first aspect of the invention, which nucleic acid sequence has the following structure:

AgB2-spacer2-TM2-endo2

AgB2 is a nucleic acid sequence encoding the antigen-binding domain of the second CAR;

spacer 2 is a nucleic acid sequence encoding the spacer of the second CAR;

TM2 is a nucleic acid sequence encoding the transmembrane domain of the second CAR;

endo 2 is a nucleic acid sequence encoding the endodomain of the second CAR.

In a fourth aspect, the present invention provides a kit comprising: a first vector which comprises the first nucleic acid sequence as defined in the third aspect; and a second vector which comprises the second nucleic acid sequence as defined in the third aspect.

The vectors may be plasmid vectors, retroviral vectors or transposon vectors. The vectors may be lentiviral vectors.

In a fifth aspect, the present invention provides a vector comprising a nucleic acid construct according to the second aspect of the invention. The vector may be a lentiviral vector.

The vector may be a plasmid vector, a retroviral vector or a transposon vector.

In a sixth aspect, the present invention provides a method for making a cell according to the first aspect of the invention, which comprises the step of introducing a nucleic acid construct according to the second aspect of the invention; one or more nucleic acid sequence(s) encoding the first and second CARs according to the third aspect of the invention; and/or a first vector and a second vector according to the fourth aspect, or a vector according to the fifth aspect, into a cell.

The cell may be from a sample isolated from a patient, a related or unrelated haematopoietic transplant donor, a completely unconnected donor, from cord blood, differentiated from an embryonic cell line, differentiated from an inducible progenitor cell line, or derived from a transformed cell line.

In an eighth aspect, the present invention provides a pharmaceutical composition comprising a plurality of cells according to the first aspect of the invention.

In a ninth aspect, the present invention provides a method for treating and/or preventing a disease, which comprises the step of administering a pharmaceutical composition according to the eighth aspect of the invention to a subject.

The method may comprise the following steps:
(i) isolation of a cell-containing sample from a subject;
(ii) transduction or transfection of the T cells with: a nucleic acid construct according to the second aspect of the invention; a first nucleic acid sequence and a second nucleic acid sequence according to the third aspect; a first vector and a second vector according to the fourth aspect or a vector according to the fifth aspect; and
(iii) administering the cells from (ii) to the subject.

The disease may be a cancer.

In a tenth aspect, the present invention provides a pharmaceutical composition according to the eighth aspect of the invention for use in treating and/or preventing a disease.

In an eleventh aspect, the present invention provides use of a T cell according to the first aspect of the invention in the manufacture of a medicament for treating and/or preventing a disease.

Alternative codons may be used in one or more portion(s) of the nucleic acid construct or the first and second nucleic acid sequences in regions which encode the same or similar amino acid sequence(s).

The logic gated CAR approach offers a significant advantage over other CAR approaches which involve targeting a single tumour-associated antigen.

A logic gate comprising a tyrosine kinase domain of CSK is advantageous over phosphatase-based approaches because CSK phosphorylation of Lck Tyr505 constitutively and fully inhibits Lck in the resting T cell state, notably before T cell activation is triggered. In contrast, phosphatases only can modify Lck in a primed state through the dephosphorylation of Tyr505 and Tyr394. This primed state of Lck is known to be partially active and requires phosphorylation from a juxtaposed Lck at Tyr394 for full activation. CSK is advantageous over a phosphatase as it locks Lck in an inhibitory state whereas phosphatases only partly inactivate Lck. The CSK inhibitory pathway mechanism of action is up-stream of dephosphorylation by phosphatases such as PTPN6/SHP-1, which signal during T cell activation, thus amplifying the inhibitory effect.

### DETAILED DESCRIPTION

Chimeric Antigen Receptors (CARs)

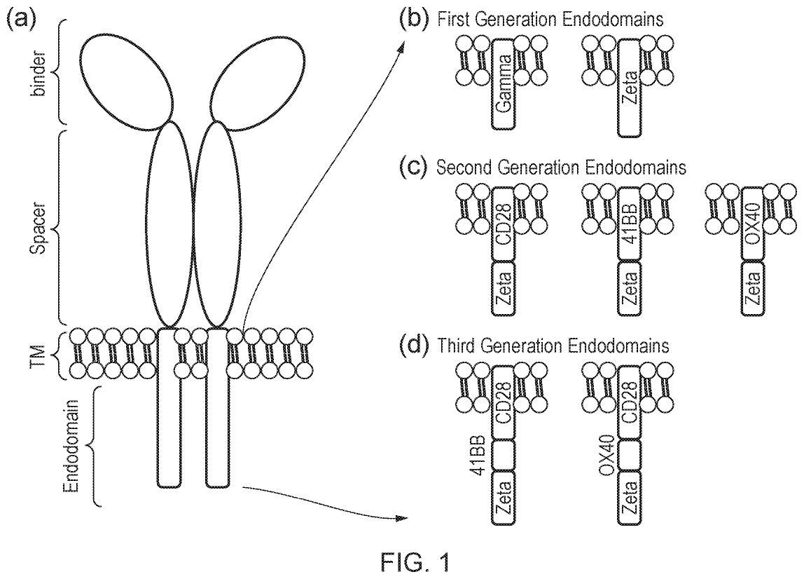

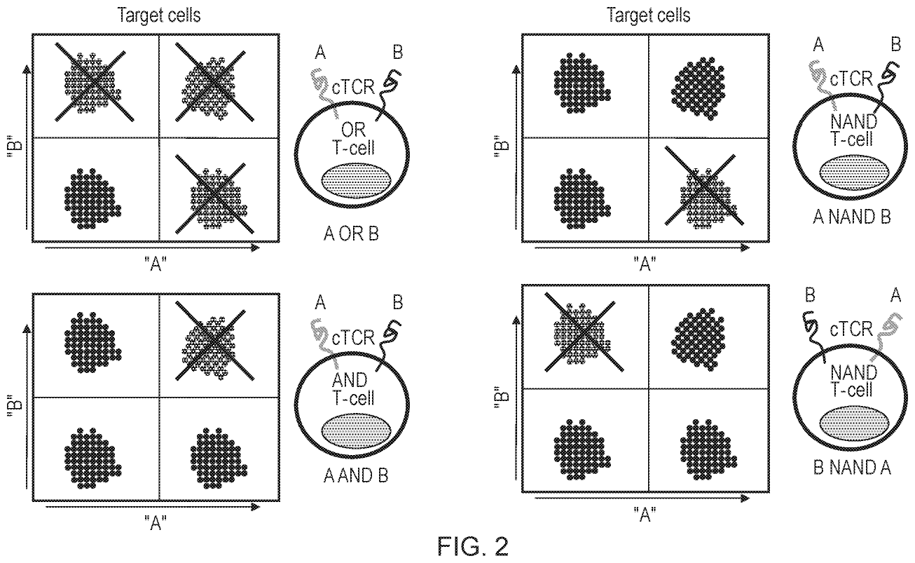

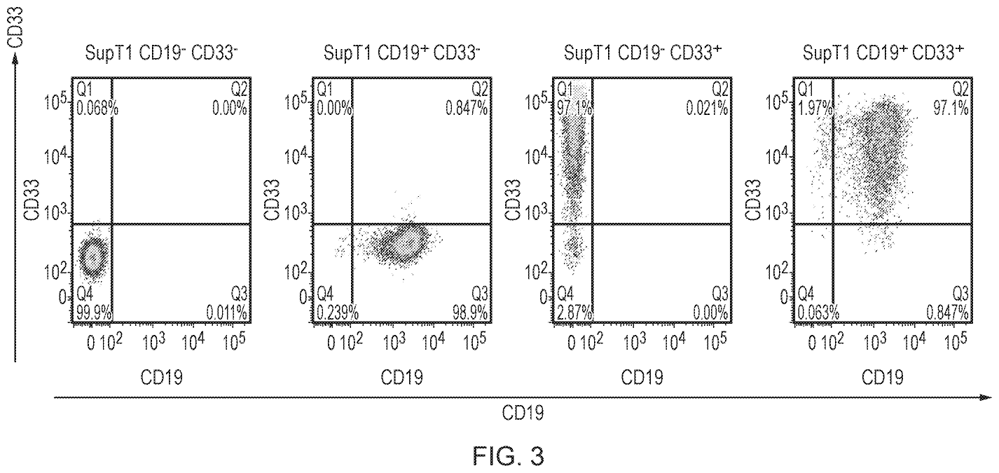

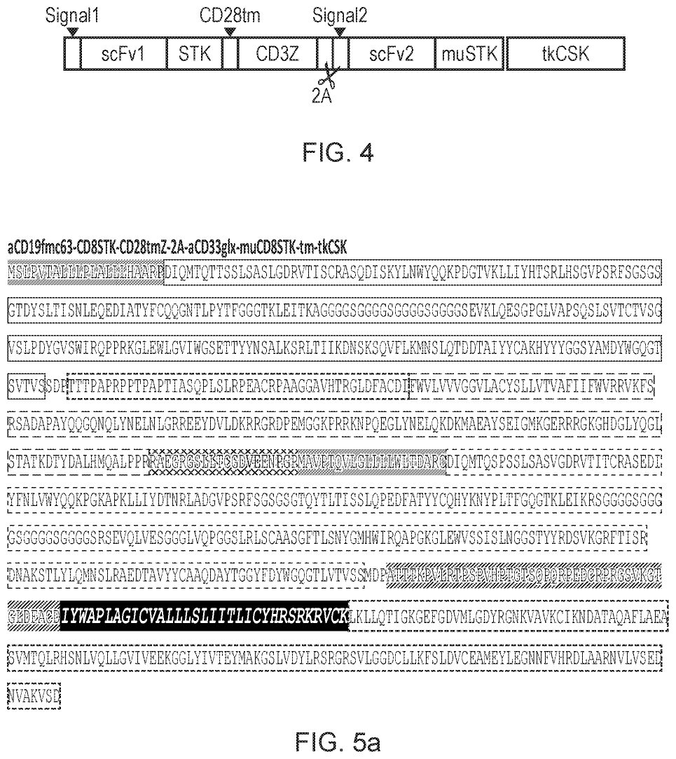

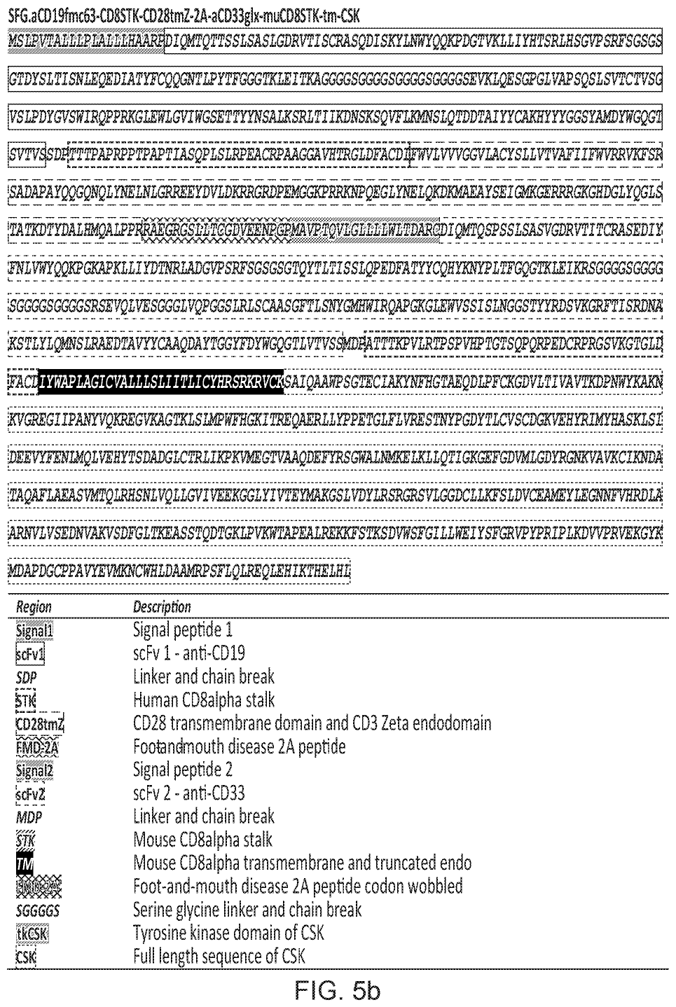

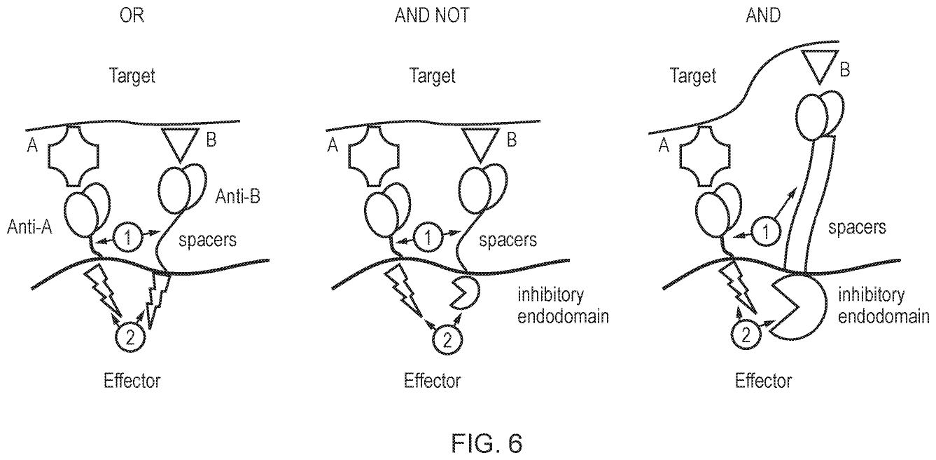

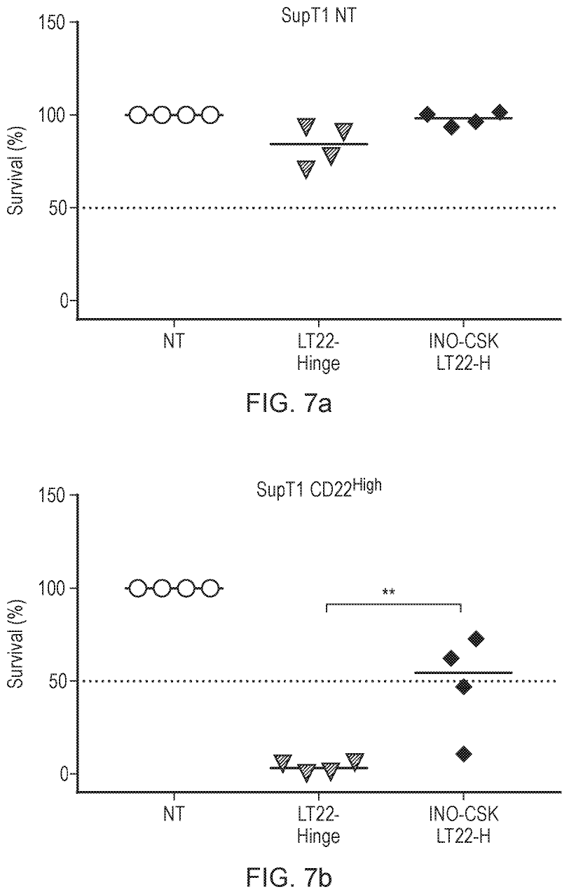

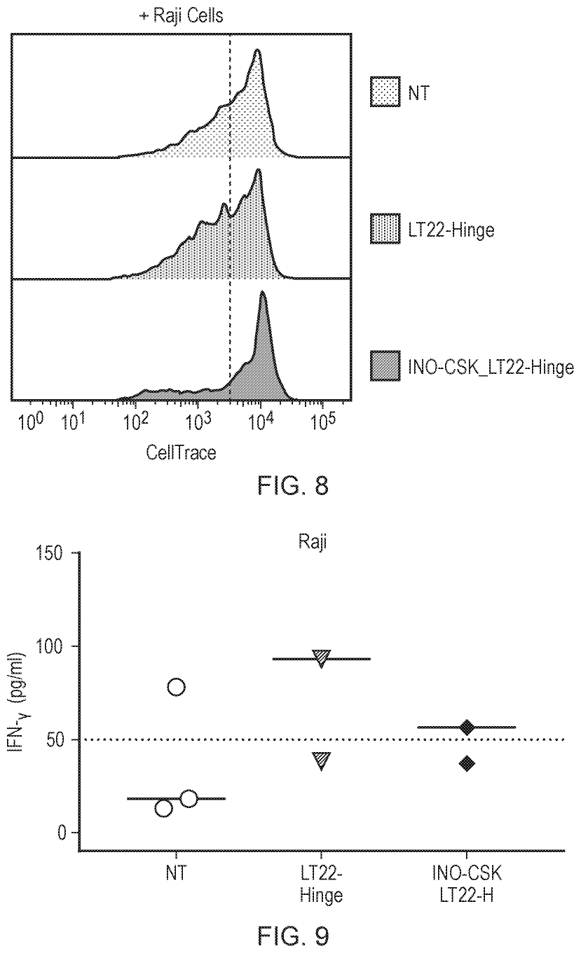

CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain. In a classical, activating CAR, the endodomain comprises an intracellular signalling domain.

Early CAR designs had endodomains derived from the intracellular parts of either the γ chain of the FcεR1 or CD3. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3t results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals, as shown in FIG. 1(d).

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen.

The first aspect of the invention relates to a cell which co-expresses a first chimeric antigen receptor (CAR) and second CAR wherein the first CAR comprises an activating endodomain and the second CAR comprises an inhibitory endodomain, wherein the inhibitory endodomain comprises a tyrosine kinase domain of C-terminal Src Kinase (CSK). The cell can recognize a desired pattern of expression on target cells in the manner of a logic gate as detailed in the truth table: table 1.

Both the first and second (and optionally subsequent) CARs may comprise:

(i) an antigen-binding domain;

(ii) a spacer;

(iii) a transmembrane domain; and

(iv) an endodomain.

TABLE 1
Truth Table for CAR AND NOT GATE

| Antigen A | Antigen B | Response |
|---|---|---|
| Absent | Absent | No activation |
| Absent | Present | No Activation |
| Present | Absent | Activation |
| Present | Present | No Activation |

The present invention also envisages a cell which coexpresses a first CAR and a second CAR, wherein the first CAR comprises the inhibitory endodomain and the second CAR comprises the activatory endodomain, wherein the inhibitory endodomain comprises a tyrosine kinase domain of C-terminal Src Kinase (CSK).

The first and second CAR of the T cell of the present invention may be produced as a polypeptide comprising both CARs, together with a cleavage site.

SEQ ID No. 1 and 2 give examples of such polypeptides, which each comprise two CARs. These sequences are annotated in FIGS. 5a and 5b.

SEQ ID No 1 encodes an activating CAR which recognizes CD19 and an inhibitory CAR which recognises CD33 and has aCSK tyrosine kinase endodomain.

SEQ ID No 2encodes an activating CAR which recognizes CD19 and an inhibitory CAR which recognises CD33 and has a full length CSK endodomain.

(CD19 CAR and CD33 CAR with CSK tyrosine kinase).
SEQ ID No. 1
MSLPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITKAGGGGSGGGGSGGGGSGGGGS
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSDPTTTPAPRPPTPAPTIASQPLSLRPEACR
PAAGGAVHTRGLDFACDIFWVLVVVGGVLACYSLLVTVAFIIFWVRRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRRAEGRGSLLTCGDVEENPGPMAVPTQVLGLLLLWLTDARCDI
QMTQSPSSLSASVGDRVTITCRASEDIYFNLVWYQQKPGKAPKLLIYDTN
RLADGVPSRFSGSGSGTQYTLTISSLQPEDFATYYCQHYKNYPLTFGQGT
KLEIKRSGGGGSGGGGSGGGGSGGGGSRSEVQLVESGGGLVQPGGSLRLS
CAASGFTLSNYGMHWIRQAPGKGLEWVSSISLNGGSTYYRDSVKGRFTIS
RDNAKSTLYLQMNSLRAEDTAVYYCAAQDAYTGGYFDYWGQGTLVTVSSM
DPATTTKPVLRTPSPVHPTGTSQPQRPEDCRPRGSVKGTGLDFACDIYWA
PLAGICVALLLSLIITLICYHRSRKRVCKLKLLQTIGKGEFGDVMLGDYR
GNKVAVKCIKNDATAQAFLAEASVMTQLRHSNLVQLLGVIVEEKGGLYIV
TEYMAKGSLVDYLRSRGRSVLGGDCLLKFSLDVCEAMEYLEGNNFVHRDL
AARNVLVSEDNVAKVSDFGLTKEASSTQDTGKLPVKWTAPEALREKKFST
KSDVWSFGILLWEIYSFGRVPYPRIPLKDVVPRVEKGYKMDAPDGCPPAV
YEVMKNCWHLDAAMRPSFLQLREQLEHIKTHELHL
(CD19 CAR and CD33 CAR with full length CSK).
SEQ ID No. 2
MSLPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITKAGGGGSGGGGSGGGGSGGGGS
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSDPTTTPAPRPPTPAPTIASQPLSLRPEACR
PAAGGAVHTRGLDFACDIFWVLVVVGGVLACYSLLVTVAFIIFWVRRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRRAEGRGSLLTCGDVEENPGPMAVPIQVLGLLLLWLTDARCDI
QMTQSPSSLSASVGDRVTITCRASEDIYFNLVWYQQKPGKAPKLLIYDTN
RLADGVPSRFSGSGSGTQYTLTISSLQPEDFATYYCQHYKNYPLTFGQGT
KLEIKRSGGGGSGGGGSGGGGSGGGGSRSEVQLVESGGGLVQPGGSLRLS
CAASGFTLSNYGMHWIRQAPGKGLEWVSSISLNGGSTYYRDSVKGRFTIS
RDNAKSTLYLQMNSLRAEDTAVYYCAAQDAYTGGYFDYWGQGTLVTVSSM
DPATTTKPVLRTPSPVHPTGTSQPQRPEDCRPRGSVKGTGLDFACDIYWA
PLAGICVALLLSLIITLICYHRSRKRVCKSAIQAAWPSGTECIAKYNFHG
TAEQDLPFCKGDVLTIVAVTKDPNWYKAKNKVGREGIIPANYVQKREGVK
AGTKLSLMPWFHGKITREQAERLLYPPETGLFLVRESTNYPGDYTLCVSC
DGKVEHYRIMYHASKLSIDEEVYFENLMQLVEHYTSDADGLCTRLIKPKV
MEGTVAAQDEFYRSGWALNMKELKLLQTIGKGEFGDVMLGDYRGNKVAVK
CIKNDATAQAFLAEASVMTQLRHSNLVQLLGVIVEEKGGLYIVTEYMAKG
SLVDYLRSRGRSVLGGDCLLKFSLDVCEAMEYLEGNNFVHRDLAARNVLV
SEDNVAKVSDFGLTKEASSTQDTGKLPVKWTAPEALREKKFSTKSDVWSF
GILLWEIYSFGRVPYPRIPLKDVVPRVEKGYKMDAPDGCPPAVYEVMKNC
WHLDAAMRPSFLQLREQLEHIKTHELHL

The CAR may comprise a variant of the CAR-encoding part of the sequence shown as SEQ ID No. 1 or 2 having at least 80, 85, 90, 95, 98 or 99% sequence identity, provided that the variant sequence is a CAR having the required properties.

Methods of sequence alignment are well known in the art and are accomplished using suitable alignment programs. The % sequence identity refers to the percentage of amino acid or nucleotide residues that are identical in the two sequences when they are optimally aligned. Nucleotide and protein sequence homology or identity may be determined using standard algorithms such as a BLAST program (Basic Local Alignment Search Tool at the National Center for Biotechnology Information) using default parameters, which is publicly available at http://blast.ncbi.nlm.nih.gov. Other algorithms for determining sequence identity or homology include: LALIGN (http://www.ebi.ac.uk/Tools/psa/lalign/ and http://www.ebi.ac.uk/Tools/psa/lalign/nucleotide.html), AMAS (Analysis of Multiply Aligned Sequences, at http://www.compbio.dundee.ac.uk/Software/Amas/amas.html), FASTA (http://www.ebi.ac.uk/Tools/sss/fasta/), Clustal Omega (http://www.ebi.ac.uk/Tools/msa/clustalo/), SIM (http://web.expasy.org/sim/), and EMBOSS Needle (http://www.ebi.ac.uk/Tools/psa/emboss_needle/nucleotide.html).

Signal Peptide

The CARs of the T cell of the present invention may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein.

The free signal peptides are then digested by specific proteases.

The signal peptide may be at the amino terminus of the molecule.

The signal peptide may comprise the SEQ ID No. 3, 4 or 5 or a variant thereof having 5, 4, 3, 2 or 1 amino acid mutations (insertions, substitutions or additions) provided that the signal peptide still functions to cause cell surface expression of the CAR.

SEQ ID No. 3:
MGTSLLCWMALCLLGADHADG

The signal peptide of SEQ ID No. 3 is compact and highly efficient. It is predicted to give about 95% cleavage after the terminal glycine, giving efficient removal by signal peptidase.

SEQ ID No. 4:
MSLPVTALLLPLALLLHAARP

The signal peptide of SEQ ID No. 4 is derived from IgG1.

SEQ ID No. 5:
MAVPTQVLGLLLLWLTDARC

The signal peptide of SEQ ID No. 5 is derived from CD8.

The signal peptide for the first CAR may have a different sequence from the signal peptide of the second CAR (and from the 3^{rd} CAR and 4^{th} CAR etc).

Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell.

The antigen binding domain may be based on a natural ligand of the antigen.

The antigen binding domain may comprise an affinity peptide from a combinatorial library or a de novo designed affinity protein/peptide.

Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

In the T cell of the present invention, the first and second CARs may comprise different spacer molecules. For example, the spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Examples of amino acid sequences for these spacers are given below:

(hinge-CH2CH3 of human IgG1)
SEQ ID No. 6
AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
SEQ ID No. 7 (human CD8 stalk):
ITTPAPRPPTPAPTIASQPLSLRPEACRPAAGGAVHIRGLDFACDI
SEQ ID No. 8 (mouse CD8a stalk):
ATTTKPVLRTPSPVHPTGTSQPQRPEDCRPRGSVKGTGLDFACD
SEQ ID No. 9 (human IgG1 hinge):
AEPKSPDKTHTCPPCPKDPK
(CD2 ectodomain)
SEQ ID No. 10
KEITNALETWGALGQDINLDIPSFQMSDDIDDIKWEKTSDKKKIAQFRKE
KETFKEKDTYKLFKNGTLKIKHLKTDDQDIYKVSIYDTKGKNVLEKIFDL
KIQERVSKPKISWTCINTTLTCEVMNGTDPELNLYQDGKHLKLSQRVITH
KWTTSLSAKFKCTAGNKVSKESSVEPVSCPEKGLD
(CD34 ectodomain)
SEQ ID no. 11
SLDNNGTATPELPTQGTFSNVSTNVSYQETTTPSTLGSTSLHPVSQHGNE
ATTNITETTVKFTSTSVITSVYGNTNSSVQSQTSVISTVFTTPANVSTPE
TTLKPSLSPGNVSDLSTTSTSLATSPTKPYTSSSPILSDIKAEIKCSGIR
EVKLTQGICLEQNKTSSCAEFKKDRGEGLARVLCGEEQADADAGAQVCSL
LLAQSEVRPQCLLLVLANRTEISSKLQLMKKHQSDLKKLGILDFTEQDVA
SHQSYSQKT

Since CARs are typically homodimers (see FIG. 1a), cross-pairing may result in a heterodimeric chimeric antigen receptor. This is undesirable for various reasons, for example: (1) the epitope may not be at the same “level” on the target cell so that a cross-paired CAR may only be able to bind to one antigen; (2) the VH and VL from the two different scFv could swap over and either fail to recognize target or worse recognize an unexpected and unpredicted antigen. For the two (or more) CARs of the cell of the present invention, the spacer of the first CAR may be sufficiently different from the spacer of the second CAR in order to avoid cross-pairing. The amino acid sequence of the first spacer may share less that 50%, 40%, 30% or 20% identity at the amino acid level with the second spacer.

An AND NOT gate requires CAR design which results in co-segregation of both CARs upon recognition of both antigens. For antigens of similar size, or for target epitopes which are a similar distance from the target cell membrane, this may be achieved using similar sized spacers. For example,pairs of orthologous spacer sequences may be employed. Examples are murine and human CD8 stalks, or alternatively spacer domains which are monomeric—for instance the ectodomain of CD2.

Examples of equal or similar sized spacer pairs are shown in the following Table:

| Stimulatory CAR spacer | Inhibitory CAR spacer |
|---|---|
| Human-CD8aSTK | Mouse CD8aSTK |
| Human-CD28STK | Mouse CD8aSTK |
| Human-IgG-Hinge | Human-CD3z ectodomain |
| Human-CD8aSTK | Mouse CD28STK |
| Human-CD28STK | Mouse CD28STK |
| Human-IgG-Hinge-CH2CH3 | Human-IgM-Hinge-CH2CH3CD4 |

All the spacer domains mentioned above form homodimers. However the mechanism is not limited to using homodimeric receptors and should work with monomeric receptors as long as the spacer is sufficiently rigid. An example of such a spacer is CD2 or truncated CD22.

An AND gate requires a design which results in kinetic segregation of the two CARs at the T-cell:target cell synapse upon recognition of both antigens. For antigens of similar size, or for target epitopes which are a similar distance from the target cell membrane, this may be achieved by choosing different spacers, one of which is longer/more bulky than the other, as described in WO2015/075469. For target epitopes which are spatially separate in terms of their distance from the target cell membrane, kinetic segregation may be achievable with similar sized spacers, as described in WO 2017/068361.

Examples of spacer pairs which have a different length and/or size are shown in the following Table:

| Stimulatory CAR spacer | Inhibitory CAR spacer |
|---|---|
| Human-CD8STK | Human-IgG-Hinge-CH2CH3 |
| Human-CD3z ectodomain | Human-IgG-Hinge-CH2CH3 |
| Human-IgG-Hinge | Human-IgG-Hinge-CH2CH3 |
| Human-CD28STK | Human-IgG-Hinge-CH2CH3 |
| Human-CD8STK | Human-IgM-Hinge-CH2CH3CD4 |
| Human-CD3z ectodomain | Human-IgM-Hinge-CH2CH3CD4 |
| Human-IgG-Hinge | Human-IgM-Hinge-CH2CH3CD4 |
| Human-CD28STK | Human-IgM-Hinge-CH2CH3CD4 |

Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes synthetic transmembrane components). The transmembrane domain may be derived from CD28, which gives good receptor stability.

Activating Endodomain

The endodomain is the signal-transmission portion of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28, OX40 and 4-1BB can be used with CD3-Zeta to transmit a proliferative/survival signal, or all three can be used together.

Where the T cell of the present invention comprises a CAR with an activating endodomain, it may comprise the CD3-Zeta endodomain alone, the CD3-Zeta endodomain with that of either CD28, OX40 or 4-1 BB or the CD28 endodomain and OX40 and CD3-Zeta endodomain and 4-1BB.

Any endodomain which contains an ITAM motif can act as an activation endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxL/I. Typically, but not always, two of these motifs are separated by between 6 and 8 amino acids in the tail of the molecule (YxxL/Ix(6-8)YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000/063372, which relates to synthetic signalling molecules).

The transmembrane and intracellular T-cell signalling domain (endodomain) of a CAR with an activating endodomain may comprise the sequence shown as SEQ ID No. 12, 13 or 14 or a variant thereof having at least 80% sequence identity.

comprising CD28 transmembrane domain and CD3 Z
endodomain
SEQ ID No. 12
FWVLVVVGGVLACYSLLVTVAFIIFWVRRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR
comprising CD28 transmembrane domain and CD28 and
CD3 Zeta endodomains
SEQ ID No. 13
FWVLVVVGGVLACYSLLVTVAFIIFWVRSKRSRLLHSDYMNMTPRRPGPT
RKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR
comprising CD28 transmembrane domain and CD28,
OX40 and CD3 Zeta endodomains.
SEQ ID No. 14
FWVLVVVGGVLACYSLLVTVAFIIFWVRSKRSRLLHSDYMNMTPRRPGPT
RKHYQPYAPPRDFAAYRSRDQRLPPDAHKPPGGGSFRTPIQEEQADAHST
LAKIRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPR

A variant sequence may have at least 80%, 85%, 90%, 95%, 98% or 99% sequence identity to SEQ ID No. 12, 13 or 14, provided that the sequence provides an effective trans-membrane domain and an effective intracellular T cell signaling domain.

Other spacers and endodomains may be tested for example using the model system exemplified herein. Target cell populations can be created by transducing a suitable cell line such as a SupT1 cell line either singly or doubly to establish cells negative for both antigens (the wild-type), positive for either and positive for both (e.g. CD19−CD33−, CD19+CD33−, CD19−CD33+ and CD19+CD33+). T cells such as the mouse T cell line BW5147 which releases IL-2 upon activation may be transduced with pairs of CARs and their ability to function in a logic gate measured through measurement of IL-2 release (for example by ELISA).

Inhibitory Endodomain

In the cell of the present invention, one of the CARs comprises an inhibitory endodomain comprising the tyrosine kinase domain of CSK.

The inhibitory endodomain may comprise all or part of a protein-tyrosine kinase CSK.

Protein tyrosine kinases (PTKs) are signaling molecules that regulate a variety of cellular processes including cell growth, differentiation, mitotic cycle, and oncogenic transformation. The N-terminal part of non-receptor (or cytoplasmic) PTK contains two tandem Src homolog (SH2) domains, which act as protein phospho-tyrosine binding domains, and mediate the interaction of this PTK with its substrates. Tyrosine proteins kinases are a subclass of protein kinase, where the phosphate group is attached to the amino acid tyrosine on the protein.

CSK (C-Terminal SRC Kinase)

Tyrosine-protein kinase CSK (C-terminal Src kinase) is an enzyme (UniProt ID: P41240 [http://www.uniprot.org/uniprot/P41240]) which phosphorylates tyrosine residues located in the C-terminal end of Src-family kinases (SFKs), such as SRC, HCK, FYN, LYN and notably LCK. CSK is mainly expressed in the lungs and macrophages as well as several other tissues. Tyrosine-kinase CSK is mainly present in the cytoplasm, but also found in lipid rafts making cell-cell junction.

CSK is a non-receptor tyrosine-protein kinase with molecular mass of 50 kDa. CSK plays an important role in the regulation of cell growth, differentiation, migration and immune response. CSK acts by suppressing the activity of the SFKs by phosphorylation of family members at a conserved C-terminal tail site.

CSK contains the SH3 and SH2 domains in its N-terminus and a kinase domain in its C-terminus. This arrangement of functional domains within the primary structure is similar to that of SFKs, but CSK lacks the N-terminal fatty acylation sites, the auto-phosphorylation site in the activation loop, and the C-terminal negative regulatory sites, all of which are conserved among SFK proteins and critical for their proper regulation. The absence of auto-phosphorylation in the activation loop is a distinguishing feature of CSK. The most striking feature of the CSK structure is that, unlike the situation in SFKs, the binding pockets of the SH3 and SH2 domains are oriented outward, enabling intermolecular interactions with other molecules. In active molecules, the SH2-kinase and SH2-SH3 linkers are tightly bound to the N-terminal lobe of the kinase domain in order to stabilize the active conformation, and there is a direct linkage between the SH2 and the kinase domains. In inactive molecules, the SH2 domains are rotated in a manner that disrupts the linkage to the kinase domain.

Upon phosphorylation by other kinases, Src-family members engage in intramolecular interactions between the phosphotyrosine tail and the SH2 domain that result in an inactive conformation. To inhibit SFKs, CSK is recruited to the plasma membrane via binding to transmembrane proteins or adapter proteins located near the plasma membrane and ultimately suppresses signaling through various surface receptors, including T-cell receptor (TCR) by phosphorylating and maintaining inactive several effector molecules.

Because Csk lacks a transmembrane domain and fatty acyl modifications, it is predominantly present in cytosol, whereas its substrate SFKs are anchored to the membrane via their N-terminal myristate and palmitate moieties. Therefore, the translocation of CSK to the membrane, where SFKs are activated, is thought to be a critical step of CSK regulation. So far, several scaffolding proteins, e.g., caveolin-1, paxillin, Dab2, VE-cadherin, IGF-1 R, IR, LIME, and SIT1, have been identified as membrane anchors of CSK, as well intrinsic phosphoprotein Cbp/PAG1 (Csk binding protein/phosphoprotein associated with glycosphingolipid-enriched membrane). Cbp has a single transmembrane domain at its N-terminus and two palmitoyl modification sites just C-terminal to the transmembrane domain, through which Cbp is exclusively localized to lipid rafts.

The present invention therefore provides a means of bringing CSK into closer proximity with SFKs (such as Lck) located at the TCR, enabling more efficient inhibition of signal transduction by SKFs in the presence of a particular antigen (A) and absence of another antigen (B) on a target cell.

The inhibitory endodomain of the CAR of the present invention may comprise all of CSK (SEQ ID No. 15) or just the tyrosine kinase domain (SEQ ID No. 16).

-sequence of full length CSK
SEQ ID No: 15
SAIQAAWPSGTECIAKYNFHGTAEQDLPFCKGDVLTIVAVTKDPNWYKAK
NKVGREGIIPANYVQKREGVKAGTKLSLMPWFHGKITREQAERLLYPPET
GLFLVRESTNYPGDYTLCVSCDGKVEHYRIMYHASKLSIDEEVYFENLMQ
LVEHYTSDADGLCTRLIKPKVMEGTVAAQDEFYRSGWALNMKELKLLQTI
GKGEFGDVMLGDYRGNKVAVKCIKNDATAQAFLAEASVMTQLRHSNLVQL
LGVIVEEKGGLYIVTEYMAKGSLVDYLRSRGRSVLGGDCLLKFSLDVCEA
MEYLEGNNFVHRDLAARNVLVSEDNVAKVSDFGLTKEASSTQDTGKLPVK
WTAPEALREKKFSTKSDVWSFGILLWEIYSFGRVPYPRIPLKDVVPRVEK
GYKMDAPDGCPPAVYEVMKNCWHLDAAMRPSFLQLREQLEHIKTHELHL
-sequence of tyrosine kinase domain of CSK
SEQ ID No: 16
LKLLQTIGKGEFGDVMLGDYRGNKVAVKCIKNDATAQAFLAEASVMTQLR
HSNLVQLLGVIVEEKGGLYIVTEYMAKGSLVDYLRSRGRSVLGGDCLLKF
SLDVCEAMEYLEGNNFVHRDLAARNVLVSEDNVAKVSDFGLTKEASSTQD
TGKLPVKWTAPEALREKKFSTKSDVWSFGILLWEIYSFGRVPYPRIPLKD
VVPRVEKGYKMDAPDGCPPAVYEVMKNCWHLDAAMRPSFLQLREQLEHIK
THELHL

The CAR of the present invention may comprise a variant of the sequence or part thereof having at least 80% sequence identity, as long as the variant retains the capacity to inhibit T cell signaling by the activating CAR.

Co-Expression Site

The second aspect of the invention relates to a nucleic acid construct which encodes the first and second CARs.

The nucleic acid construct may produce a polypeptide which comprises the two CAR molecules joined by a cleavage site. The cleavage site may be self-cleaving, such that when the polypeptide is produced, it is immediately cleaved into the first and second CARs without the need for any external cleavage activity.

Various self-cleaving sites are known, including the Foot-and-Mouth disease virus (FMDV) 2a self-cleaving peptide, which has the sequence shown as SEQ ID No. 17:

SEQ ID No. 17
RAEGRGSLLTCGDVEENPGP.

The co-expressing sequence may be an internal ribosome entry sequence (IRES). The co-expressing sequence may be an internal promoter.

Cell

The first aspect of the invention relates to a cell which co-expresses a first CAR and a second CAR at the cell surface.

The cell may be any eukaryotic cell capable of expressing a CAR at the cell surface, such as an immunological cell.

In particular the cell may be an immune effector cell such as a T cell or a natural killer (NK) cell

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The T cell of the invention may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

CAR-expressing cells, such as CAR-expressing T or NK cells, may either be created ex vivo either from a patient's own peripheral blood (1^{st} party), or in the setting of a haematopoietic stem cell transplant from donor peripheral blood (2^{nd} party), or peripheral blood from an unconnected donor (3^{rd} party).

The present invention also provide a cell composition comprising CAR expressing T cells and/or CAR expressing NK cells according to the present invention. The cell composition may be made by tranducing or transfecting a blood-sample ex vivo with a nucleic acid according to the present invention.

Alternatively, CAR-expressing cells may be derived from ex vivo differentiation of inducible progenitor cells or embryonic progenitor cells to the relevant cell type, such as T cells. Alternatively, an immortalized cell line such as a T-cell line which retains its lytic function and could act as a therapeutic may be used.

In all these embodiments, CAR cells are generated by introducing DNA or RNA coding for the CARs by one of many means including transduction with a viral vector, transfection with DNA or RNA.

A CAR T cell of the invention may be an ex vivo T cell from a subject. The T cell may be from a peripheral blood mononuclear cell (PBMC) sample. T cells may be activated and/or expanded prior to being transduced with CAR-encoding nucleic acid, for example by treatment with an anti-CD3 monoclonal antibody.

A CAR T cell of the invention may be made by:
(i) isolation of a T cell-containing sample from a subject or other sources listed above; and
(ii) transduction or transfection of the T cells with one or more nucleic acid sequence(s) encoding the first and second CAR.

The T cells may then by purified, for example, selected on the basis of co-expression of the first and second CAR.

Nucleic Acid Sequences

The second aspect of the invention relates to one or more nucleic acid sequence(s) which codes for a first CAR and a second CAR as defined in the first aspect of the invention.

The nucleic acid sequence may comprise one of the following sequences, or a variant thereof:

Dual CAR system using tyrosine kinase domain CSK
as endodomain (CD19 and CD33)
SEQ ID No: 18
ATGAGCCTGCCCGTGACCGCCCTGCTGCTGCCCCTGGCCCTGCTGCTGCA
CGCCGCCAGACCAGACATCCAGATGACCCAGACCACCAGCAGCCTGAGCG
CCAGCCTGGGCGACCGGGTGACCATCAGCTGCAGAGCCAGCCAGGACATC
AGCAAGTACCTGAACTGGTACCAGCAGAAGCCCGACGGCACCGTGAAGCT
GCTGATCTACCACACCAGCCGGCTGCACAGCGGCGTGCCCAGCCGGTTCA
GCGGCAGCGGCAGCGGCACCGACTACAGCCTGACCATCAGCAACCTGGAG
CAGGAGGACATCGCCACCTACTTCTGCCAGCAGGGCAACACCCTGCCCTA
CACCTTCGGAGGCGGCACCAAGCTGGAGATCACCAAGGCCGGAGGCGGAG
GCTCTGGCGGAGGCGGCTCTGGCGGAGGCGGCTCTGGCGGAGGCGGCAGC
GAGGTGAAGCTGCAGGAGTCTGGCCCAGGCCTGGTGGCCCCAAGCCAGAG
CCTGAGCGTGACCTGCACCGTGAGCGGCGTGAGCCTGCCCGACTACGGCG
TGAGCTGGATCAGGCAGCCCCCACGGAAGGGCCTGGAGTGGCTGGGCGTG
ATCTGGGGCAGCGAGACCACCTACTACAACAGCGCCCTGAAGAGCCGGCT
GACCATCATCAAGGACAACAGCAAGAGCCAGGTGTTCCTGAAGATGAACA
GCCTGCAGACCGACGACACCGCCATCTACTACTGCGCCAAGCACTACTAC
TATGGCGGCAGCTACGCTATGGACTACTGGGGCCAGGGCACCAGCGTGAC
CGTGAGCTCAGATCCCACCACGACGCCAGCGCCGCGACCACCAACACCGG
CGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGA
TATCTTTTGGGTGCTGGTGGTGGTTGGTGGAGTCCTGGCTTGCTATAGCT
TGCTAGTAACAGTGGCCTTTATTATTTTCTGGGTGAGGAGAGTGAAGTTC
AGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGA
GACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTA
CAGTGAGATTGGGATGAAAGGCGAGCGCCGGAGGGGCAAGGGGCACGATG
GCCTTTACCAGGGTCTCAGTACAGCCACCAAGGACACCTACGACGCCCTT
CACATGCAGGCCCTGCCTCCTCGCAGAGCCGAGGGCAGGGGAAGTCTTCT
AACATGCGGGGACGTGGAGGAAAATCCCGGGCCCATGGCCGTGCCCACTC
AGGTCCTGGGGTTGTTGCTACTGTGGCTTACAGATGCCAGATGTGACATC
CAGATGACACAGTCTCCATCTTCCCTGTCTGCATCTGTCGGAGATCGCGT
CACCATCACCTGTCGAGCAAGTGAGGACATTTATTTTAATTTAGTGTGGT
ATCAGCAGAAACCAGGAAAGGCCCCTAAGCTCCTGATCTATGATACAAAT
CGCTTGGCAGATGGGGTCCCATCACGGTTCAGTGGCTCTGGATCTGGCAC
ACAGTATACTCTAACCATAAGTAGCCTGCAACCCGAAGATTTCGCAACCT
ATTATTGTCAACACTATAAGAATTATCCGCTCACGTTCGGTCAGGGGACC
AAGCTGGAAATCAAAAGATCTGGTGGCGGAGGGTCAGGAGGCGGAGGCAG
CGGAGGCGGTGGCTCGGGAGGCGGAGGCTCGAGATCTGAGGTGCAGTTGG
TGGAGTCTGGGGGCGGCTTGGTGCAGCCTGGAGGGTCCCTGAGGCTCTCC
TGTGCAGCCTCAGGATTCACTCTCAGTAATTATGGCATGCACTGGATCAG
GCAGGCTCCAGGGAAGGGTCTGGAGTGGGTCTCGTCTATTAGTCTTAATG
GTGGTAGCACTTACTATCGAGACTCCGTGAAGGGCCGATTCACTATCTCC
AGGGACAATGCAAAAAGCACCCTCTACCTTCAAATGAATAGTCTGAGGGC
CGAGGACACGGCCGTCTATTACTGTGCAGCACAGGACGCTTATACGGGAG
GTTACTTTGATTACTGGGGCCAAGGAACGCTGGTCACAGTCTCGTCTATG
GATCCCGCCACCACAACCAAGCCCGTGCTGCGGACCCCAAGCCCTGTGCA
CCCTACCGGCACCAGCCAGCCTCAGAGACCCGAGGACTGCCGGCCTCGGG
GCAGCGTGAAGGGCACCGGCCTGGACTTCGCCTGCGACATCTACTGGGCA
CCTCTGGCCGGAATATGCGTGGCACTGCTGCTGAGCCTCATCATCACCCT
GATCTGTTATCACCGAAGCCGCAAGCGGGTGTGTAAACTGAAGCTGCTGC
AGACCATCGGCAAGGGCGAGTTTGGAGATGTGATGCTGGGCGACTACCGG
GGCAACAAGGTGGCAGTGAAGTGCATCAAGAACGACGCTACAGCCCAGGC
TTTTCTGGCCGAAGCCAGCGTGATGACCCAGCTGAGACACAGCAATCTGG
TGCAGCTGCTGGGCGTGATCGTGGAAGAAAAAGGCGGCCTGTATATCGTG
ACCGAGTACATGGCCAAGGGCAGCCTGGTGGACTACCTGAGAAGTAGAGG
CAGAAGCGTGCTCGGAGGCGACTGCCTGCTGAAGTTTAGCCTGGATGTGT
GCGAGGCTATGGAATACCTGGAAGGCAACAACTTCGTGCACCGCGATCTG
GCCGCCAGAAATGTGCTGGTGTCCGAGGACAACGTGGCCAAGGTGTCCGA
TTTCGGCCTGACCAAAGAGGCCAGCAGCACCCAGGATACAGGCAAGCTGC
CCGTGAAATGGACAGCCCCTGAGGCTCTGAGAGAGAAGAAGTTCAGCACC
AAGAGCGACGTGTGGTCCTTCGGCATCCTGCTGTGGGAAATCTACAGCTT
CGGCAGAGTGCCCTATCCTAGAATCCCTCTGAAGGACGTGGTGCCCAGAG
TGGAAAAGGGCTACAAGATGGATGCCCCTGACGGCTGTCCTCCTGCCGTG
TACGAAGTGATGAAGAACTGCTGGCACCTGGACGCCGCTATGAGGCCATC
TTTCCTGCAGCTGAGAGAGCAGCTGGAACACATCAAGACCCACGAGCTGC
ACCTG
Dual CAR system using full sequence CSK as
endodomain (CD19 and CD33)
SEQ ID No: 19
ATGAGCCTGCCCGTGACCGCCCTGCTGCTGCCCCTGGCCCTGCTGCTGCA
CGCCGCCAGACCAGACATCCAGATGACCCAGACCACCAGCAGCCTGAGCG
CCAGCCTGGGCGACCGGGTGACCATCAGCTGCAGAGCCAGCCAGGACATC
AGCAAGTACCTGAACTGGTACCAGCAGAAGCCCGACGGCACCGTGAAGCT
GCTGATCTACCACACCAGCCGGCTGCACAGCGGCGTGCCCAGCCGGTTCA
GCGGCAGCGGCAGCGGCACCGACTACAGCCTGACCATCAGCAACCTGGAG
CAGGAGGACATCGCCACCTACTTCTGCCAGCAGGGCAACACCCTGCCCTA
CACCTTCGGAGGCGGCACCAAGCTGGAGATCACCAAGGCCGGAGGCGGAG
GCTCTGGCGGAGGCGGCTCTGGCGGAGGCGGCTCTGGCGGAGGCGGCAGC
GAGGTGAAGCTGCAGGAGTCTGGCCCAGGCCTGGTGGCCCCAAGCCAGAG
CCTGAGCGTGACCTGCACCGTGAGCGGCGTGAGCCTGCCCGACTACGGCG
TGAGCTGGATCAGGCAGCCCCCACGGAAGGGCCTGGAGTGGCTGGGCGTG
ATCTGGGGCAGCGAGACCACCTACTACAACAGCGCCCTGAAGAGCCGGCT
GACCATCATCAAGGACAACAGCAAGAGCCAGGTGTTCCTGAAGATGAACA
GCCTGCAGACCGACGACACCGCCATCTACTACTGCGCCAAGCACTACTAC
TATGGCGGCAGCTACGCTATGGACTACTGGGGCCAGGGCACCAGCGTGAC
CGTGAGCTCAGATCCCACCACGACGCCAGCGCCGCGACCACCAACACCGG
CGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGA
TATCTTTTGGGTGCTGGTGGTGGTTGGTGGAGTCCTGGCTTGCTATAGCT
TGCTAGTAACAGTGGCCTTTATTATTTTCTGGGTGAGGAGAGTGAAGTTC
AGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGA
GACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTA
CAGTGAGATTGGGATGAAAGGCGAGCGCCGGAGGGGCAAGGGGCACGATG
GCCTTTACCAGGGTCTCAGTACAGCCACCAAGGACACCTACGACGCCCTT
CACATGCAGGCCCTGCCTCCTCGCAGAGCCGAGGGCAGGGGAAGTCTTCT
AACATGCGGGGACGTGGAGGAAAATCCCGGGCCCATGGCCGTGCCCACTC
AGGTCCTGGGGTTGTTGCTACTGTGGCTTACAGATGCCAGATGTGACATC
CAGATGACACAGTCTCCATCTTCCCTGTCTGCATCTGTCGGAGATCGCGT
CACCATCACCTGTCGAGCAAGTGAGGACATTTATTTTAATTTAGTGTGGT
ATCAGCAGAAACCAGGAAAGGCCCCTAAGCTCCTGATCTATGATACAAAT
CGCTTGGCAGATGGGGTCCCATCACGGTTCAGTGGCTCTGGATCTGGCAC
ACAGTATACTCTAACCATAAGTAGCCTGCAACCCGAAGATTTCGCAACCT
ATTATTGTCAACACTATAAGAATTATCCGCTCACGTTCGGTCAGGGGACC
AAGCTGGAAATCAAAAGATCTGGTGGCGGAGGGTCAGGAGGCGGAGGCAG
CGGAGGCGGTGGCTCGGGAGGCGGAGGCTCGAGATCTGAGGTGCAGTTGG
TGGAGTCTGGGGGCGGCTTGGTGCAGCCTGGAGGGTCCCTGAGGCTCTCC
TGTGCAGCCTCAGGATTCACTCTCAGTAATTATGGCATGCACTGGATCAG
GCAGGCTCCAGGGAAGGGTCTGGAGTGGGTCTCGTCTATTAGTCTTAATG
GTGGTAGCACTTACTATCGAGACTCCGTGAAGGGCCGATTCACTATCTCC
AGGGACAATGCAAAAAGCACCCTCTACCTTCAAATGAATAGTCTGAGGGC
CGAGGACACGGCCGTCTATTACTGTGCAGCACAGGACGCTTATACGGGAG
GTTACTTTGATTACTGGGGCCAAGGAACGCTGGTCACAGTCTCGTCTATG
GATCCCGCCACCACAACCAAGCCCGTGCTGCGGACCCCAAGCCCTGTGCA
CCCTACCGGCACCAGCCAGCCTCAGAGACCCGAGGACTGCCGGCCTCGGG
GCAGCGTGAAGGGCACCGGCCTGGACTTCGCCTGCGACATCTACTGGGCA
CCTCTGGCCGGAATATGCGTGGCACTGCTGCTGAGCCTCATCATCACCCT
GATCTGTTATCACCGAAGCCGCAAGCGGGTGTGTAAAAGCGCCATTCAGG
CCGCTTGGCCTTCTGGCACAGAGTGTATCGCCAAGTACAACTTCCACGGC
ACCGCCGAGCAGGACCTGCCTTTCTGTAAAGGCGACGTGCTGACCATCGT
GGCCGTGACAAAGGACCCCAACTGGTACAAGGCCAAGAACAAAGTGGGCA
GAGAGGGCATCATCCCCGCCAACTATGTGCAGAAGAGAGAGGGCGTTAAG
GCCGGCACCAAGCTGTCTCTGATGCCCTGGTTTCACGGCAAGATCACCAG
AGAGCAGGCCGAGAGACTGCTGTACCCTCCTGAAACCGGCCTGTTCCTCG
TGCGCGAGAGCACAAATTACCCCGGCGACTACACCCTGTGTGTGTCCTGT
GATGGCAAGGTGGAACACTACCGGATCATGTACCACGCCAGCAAGCTGAG
CATCGACGAGGAAGTGTACTTCGAGAACCTGATGCAGCTGGTCGAGCACT
ACACCTCCGATGCCGATGGCCTGTGCACCAGACTGATCAAGCCCAAAGTG
ATGGAAGGCACCGTGGCCGCTCAGGACGAGTTTTACAGATCCGGCTGGGC
TCTGAACATGAAGGAACTGAAGCTGCTGCAGACCATCGGCAAGGGCGAGT
TTGGAGATGTGATGCTGGGCGACTACCGGGGCAACAAGGTGGCAGTGAAG
TGCATCAAGAACGACGCTACAGCCCAGGCTTTTCTGGCCGAAGCCAGCGT
GATGACCCAGCTGAGACACAGCAATCTGGTGCAGCTGCTGGGCGTGATCG
TGGAAGAAAAAGGCGGCCTGTATATCGTGACCGAGTACATGGCCAAGGGC
AGCCTGGTGGACTACCTGAGAAGTAGAGGCAGAAGCGTGCTCGGAGGCGA
CTGCCTGCTGAAGTTTAGCCTGGATGTGTGCGAGGCTATGGAATACCTGG
AAGGCAACAACTTCGTGCACCGCGATCTGGCCGCCAGAAATGTGCTGGTG
TCCGAGGACAACGTGGCCAAGGTGTCCGATTTCGGCCTGACCAAAGAGGC
CAGCAGCACCCAGGATACAGGCAAGCTGCCCGTGAAATGGACAGCCCCTG
AGGCTCTGAGAGAGAAGAAGTTCAGCACCAAGAGCGACGTGTGGTCCTTC
GGCATCCTGCTGTGGGAAATCTACAGCTTCGGCAGAGTGCCCTATCCTAG
AATCCCTCTGAAGGACGTGGTGCCCAGAGTGGAAAAGGGCTACAAGATGG
ATGCCCCTGACGGCTGTCCTCCTGCCGTGTACGAAGTGATGAAGAACTGC
TGGCACCTGGACGCCGCTATGAGGCCATCTTTCCTGCAGCTGAGAGAGCA
GCTGGAACACATCAAGACCCACGAGCTGCACCTG

The nucleic acid sequence may encode the same amino acid sequence as that encoded by SEQ ID No. 18 but may have a different nucleic acid sequence, due to the degeneracy of the genetic code. The nucleic acid sequence may have at least 80, 85, 90, 95, 98 or 99% identity to the sequence shown as SEQ ID No. 18 provided that it encodes a first CAR and a second CAR as defined in the first aspect of the invention.

Vector

The present invention also provides a vector, or kit of vectors which comprises one or more CAR-encoding nucleic acid sequence(s). Such a vector may be used to introduce the nucleic acid sequence(s) into a host cell so that it expresses the first and second CARs.

The vector may, for example, be a plasmid or a viral vector, such as a retroviral vector or a lentiviral vector, or a transposon based vector or synthetic mRNA.

The vector may be capable of transfecting or transducing a T cell.

Pharmaceutical Composition

The present invention also relates to a pharmaceutical composition containing a plurality of CAR-expressing cells, such as T cells or NK cells, according to the first aspect of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The T cells of the present invention may be capable of killing target cells, such as cancer cells. The target cell may be recognisable by a defined pattern of antigen expression, for example the expression of antigen A AND antigen B; antigen A AND NOT antigen B; or a complex iteration of these gates.

T cells of the present invention may be used for the treatment of an infection, such as a viral infection.

T cells of the invention may also be used for the control of pathogenic immune responses, for example in autoimmune diseases, allergies and graft-vs-host rejection.

T cells of the invention may be used for the treatment of a cancerous disease, such as bladder cancer, breast cancer, colon cancer, endometrial cancer, kidney cancer (renal cell), leukemia, lung cancer, melanoma, non-Hodgkin lymphoma, pancreatic cancer, prostate cancer and thyroid cancer.

It is particularly suited for treatment of solid tumours where the availability of good selective single targets is limited.

T cells of the invention may be used to treat: cancers of the oral cavity and pharynx which includes cancer of the tongue, mouth and pharynx; cancers of the digestive system which includes oesophageal, gastric and colorectal cancers; cancers of the liver and biliary tree which includes hepatocellular carcinomas and cholangiocarcinomas; cancers of the respiratory system which includes bronchogenic cancers and cancers of the larynx; cancers of bone and joints which includes osteosarcoma; cancers of the skin which includes melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers including gliomas, glioblastoma multiforme and medullobastomas; cancers of the endocrine system including thyroid cancer, adrenal carcinoma and cancers associated with multiple endocrine neoplasm syndromes; lymphomas including Hodgkin's lymphoma and non-Hodgkin lymphoma; Multiple Myeloma and plasmacytomas; leukaemias both acute and chronic, myeloid or lymphoid; and cancers of other and unspecified sites including neuroblastoma.

Treatment with the T cells of the invention may help prevent the escape or release of tumour cells which often occurs with standard approaches.

The invention will now be further described by way of Examples, which are meant to serve to assist one of ordinary skill in the art in carrying out the invention and are not intended in any way to limit the scope of the invention.

### EXAMPLES

### Example 1

### Creation of Target Cell Populations

For the purposes of proving the principle of the invention, receptors based on anti-CD19 and anti-CD33 were arbitrarily chosen. Using retroviral vectors, CD19 and CD33 were cloned. These proteins were truncated so that they do not signal and could be stably expressed for prolonged periods. Next, these vectors were used to transduce the SupT1 cell line either singly or doubly to establish cells negative for both antigen (the wild-type), positive for either and positive for both. The expression data are shown in FIG. 3.

### Example 2

### Design of a Dual CAR System

A dual CAR system was designed as follows: two CARs co-expressed whereby the first recognizes CD19, has a human CD8 stalk spacer and an activating endodomain; co-expressed with an anti-CD33 CAR with a mouse CD8 stalk spacer and an endodomain comprising of the tyrosine kinase domain of CSK (SEQ ID NO: 1 and 2, FIGS. 5a and 5b). A suitable cassette is shown in FIG. 4, and a schematic of the AND NOT gate system is shown in FIG. 6.

### Example 3

### Investigating the Effect of the CSK Endodomain on T Cell Signalling

a) FACs-Based Killing (FBK)

CARs were created with and without CSK endodomains and their cytotoxic capability was compared. The CAR system tested comprised a first CAR comprising an CD22 antigen binding domain derived from Inotuzumab (INO) and a second CAR with an LT22 antigen binding domain CAR. The INO scFv tested was the clone g5/44. The CSK CARs tested comprised the INO scFv, a CD8stalk spacer, a transmembrane domain, and the intracellular domain comprising a tyrosine kinase domain of CSK.

Seven days after the thawing of PBMCs, the culture was depleted of CD56 NK cells to reduce background cytotoxicity. On the eighth day, the T-cells were co-cultured with the target cells at a ratio 1:1. The assay was carried out in a 96-well plate in 0.2 ml total volume using 5×10⁴ transduced T-cells per well and an equal number of target cells. The co-cultures are set up after being normalised for the transduction efficiency. The FBK was carried out after 72 h of incubation.

The results of the FBK are shown in FIGS. 7a and 7b for SupT1 and SupT1 CD22 cells, respectively. It is clear that cells co-expressing one CAR comprising a CSK endodomain with another CAR comprising an activating endodomain are inferior at killing than a CAR construct without such a CSK endodomain. For example, the LT22-Hinge CAR, which lacks a CSK endodomain, shows significantly lower overall cell survival than the CAR construct comprising a CSK endodomain.

b) Proliferation Assay (PA)

Proliferation is a key feature of CAR-mediated responses which is measured to test the efficacy of a CAR alongside cytotoxicity and cytokine secretion. Although 1^{st} generation CARs display good levels of cytotoxicity, they do not display good proliferative responses in vitro and fail to persist well in vivo. Proliferation is enhanced by the inclusion of co-stimulatory domains such as CD28, OX40 or 4-1BB into the CAR endodomain.

In order to measure proliferation, CD56-depleted, the same CAR-expressing T cells described in Example 3(a) were labelled with the dye Cell Trace Violet (CTV), a fluorescent dye which is hydrolysed and retained within the cell. It is excited by the 405 nm (violet) laser and fluorescence can be detected in the pacific blue channel. The CTV dye was reconstituted to 5 mM in DMSO. The T-cells were resuspended at 2×10⁶ cells per ml in PBS, and 1 ul/ml of CTV was added. The T-cells were incubated the CTV for 20 minutes at 37° C. Subsequently, the cells were quenched by adding 5 mL of complete media. After a 5 minutes incubation, the T-cells were washed and resuspended in 2 ml of complete media. An additional 10 minute incubation at room temperature allowed the occurrence of acetate hydrolysis and retention of the dye.

Labelled T-cells were co-cultured with antigen-expressing or antigen-negative target cells for seven days. The assay was carried out in a 96-well plate in 0.2 ml total volume using 5×10⁴ transduced T-cells per well and an equal number of target cells (ratio 1:1). At the day seven time point, the T-cells were analysed by flow cytometry to measure the dilution of the CTV which occurs as the T-cells divide.

FIG. 8 shows that CAR constructs comprising a CSK endodomain demonstrate decreased proliferation compared to constructs lacking the CSK endodomain: the area under the curve in the INO-CSK_LT22-Hinge CAR construct has shifted least along the X-axis compared to the LT22-Hinge CAR construct.

c) Cytokine Bead Array (CBA)

Typically, immune cells detect major histocompatibility complex (MHC) presented on infected cell surfaces, triggering cytokine release, causing lysis or apoptosis. Cytokine production by CAR T cells can activate host immunity and represent a key element as to why these effector cells are successful. Cytokines such as IFN-γ from CAR cells also recruit and activate a variety of host immune cells to modulate the tumour microenvironment and disrupt tumour growth. Therefore to test the effectivity of the CAR constructs the inventors also chose to compare IFN-γ cytokine production.

CAR constructs described in Example 3(a) were compared for IFN-γ secretion (FIG. 9) after 72 hours co-culture with Raji target cells. Decreased cytokine production was observed in the CAR constructs comprising a CSK endodomain (INO-CSK LT22-H) compared to constructs lacking a CSK endodomain (e.g.LT22-Hinge).

These data demonstrate the capacity of a CSK endodomain to inhibit of T cell activation in terms of reduced cytotoxicity, proliferation and cytokine production.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cell biology or related fields are intended to be within the scope of the following claims.

1. A cell which co-expresses a first chimeric antigen receptor (CAR) and second CAR wherein the first CAR comprises an activating endodomain and the second CAR comprises an inhibitory endodomain, wherein the inhibitory endodomain comprises tyrosine kinase domain of C-terminal Src Kinase (CSK).

2. A cell according to claim 1, wherein each CAR comprises (i) an antigen binding domain, (ii) a spacer, (iii) a trans-membrane domain, and (iv) an endodomain.

3. A cell according to claim 2, wherein the spacers of the first and second CARs are orthologous.

4. A cell according to claim 1, wherein the inhibitory endodomain comprises the amino acid sequence SEQ ID NO: 15 or SEQ ID NO: 16.

5. A cell according to claim 2, wherein the first CAR comprises an antigen-binding domain which binds CD33 and the second CAR comprises an antigen-binding domain which binds CD34.

6. A nucleic acid construct encoding a first CAR and a second CAR wherein the first CAR comprises an activating endodomain and the second CAR comprises an inhibitory endodomain, wherein the inhibitory endodomain comprises tyrosine kinase domain of C-terminal Src Kinase (CSK).

7. A nucleic acid construct according to claim 6, which has the following structure:
AgB1-spacer1-TM1-endo1-coexpr-AbB2-spacer2-TM2-endo2
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the activating endodomain of the first CAR;
coexpr is a nucleic acid sequence enabling co-expression of both CARs
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the inhibitory endodomain of the second CAR;
which nucleic acid sequence, when expressed in a cell, encodes a polypeptide which is cleaved at the cleavage site such that the first and second CARs are co-expressed at the cell surface.

8. A nucleic acid construct according to claim 7, wherein coexpr encodes a sequence comprising a self-cleaving peptide.

9. A nucleic acid construct according to claim 7, wherein alternative codons are used in regions of sequence encoding the same or similar amino acid sequences, in order to avoid homologous recombination.

10. A kit which comprises
(i) a first nucleic acid sequence or vector encoding a first CAR which comprises an activating endodomain, which nucleic acid sequence has the following structure:
AgB1-spacer1-TM1 -endo1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the activating endodomain of the first CAR; and
(ii) a second nucleic acid sequence or vector encoding a second CAR which comprises an inhibitory endodomain, wherein the inhibitory endodomain comprises tyrosine kinase domain of C-terminal Src Kinase (CSK), which nucleic acid sequence has the following structure:
AgB2-spacer2-TM2-endo2
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the inhibitory endodomain of the second CAR.

11. (canceled)

12. A kit according to claim 10, wherein the vectors are integrating viral vectors or transposons.

13. A vector comprising a nucleic acid construct according to claim 6.

14. A retroviral vector or a lentiviral vector or a transposon according to claim 13.

15. A method for making a cell according to claim 1, which comprises the step of introducing into a cell:
a) a nucleic acid construct encoding a first CAR and a second CAR wherein the first CAR comprises an activating endodomain and the second CAR comprises an inhibitory endodomain, wherein the inhibitory endodomain comprises tyrosine kinase domain of C-terminal Src Kinase (CSK);
b) a first nucleic acid sequence and a second nucleic acid sequence, wherein
(i) the first nucleic acid sequence encodes a first CAR which comprises an activating endodomain, which nucleic acid sequence has the following structure:
AgB1-spacer1-TM1-endo1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the activating endodomain of the first CAR; and
(ii) the second nucleic acid sequence encodes a second CAR which comprises an inhibitory endodomain, wherein the inhibitory endodomain comprises tyrosine kinase domain of C-terminal Src Kinase (CSK), which nucleic acid sequence has the following structure:
AgB2-spacer2-TM2-endo2
In which
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the inhibitory endodomain of the second CAR; or
c) a first vector and a second vector, wherein
(i) the first vector comprises a first nucleic acid sequence encoding a first CAR which comprises an activating endodomain, which nucleic acid sequence has the following structure:
AgB1-spacer1-TM1-endo1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the activating endodomain of the first CAR; and
(ii) the second vector comprises a second nucleic acid sequence encoding a second CAR which comprises an inhibitory endodomain, wherein the inhibitory endodomain comprises tyrosine kinase domain of C-terminal Src Kinase (CSK), which nucleic acid sequence has the following structure:
AgB2-spacer2-TM2-endo2
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the inhibitory endodomain of the second CAR.

16. A method according to claim 15, wherein the cell is from a sample isolated from a subject.

17. A pharmaceutical composition comprising a plurality of cells according to claim 1.

18. A method for treating and/or preventing a disease, which comprises the step of administering a pharmaceutical composition according to claim 17 to a subject.

19. A method according to claim 18, which comprises the following steps:
(i) isolation of a cell-containing sample from a subject;
(ii) transduction or transfection of the cells with:
a) a nucleic acid construct or vector encoding a first CAR and a second CAR wherein the first CAR comprises an activating endodomain and the second CAR comprises an inhibitory endodomain, wherein the inhibitory endodomain comprises tyrosine kinase domain of C-terminal Src Kinase (CSK);according to any of claims 6 to 9;
b) a first nucleic acid sequence and a second nucleic acid sequence, wherein
the first nucleic acid sequence encodes a first CAR which comprises an activating endodomain, which nucleic acid sequence has the following structure:
AgB1-spacer1-TM1-endo1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the activating endodomain of the first CAR; and
the second nucleic acid sequence encodes a second CAR which comprises an inhibitory endodomain, wherein the inhibitory endodomain comprises tyrosine kinase domain of C-terminal Src Kinase (CSK), which nucleic acid sequence has the following structure:
AgB2-spacer2-TM2-endo2
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the inhibitory endodomain of the second CAR; or
c) a first vector and a second vector, wherein
the first vector comprises a first nucleic acid sequence encoding a first CAR which comprises an activating endodomain, which nucleic acid sequence has the following structure:
AgB1-spacer1-TM1-endo1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the activating endodomain of the first CAR; and
the second vector comprises a second nucleic acid sequence encoding a second CAR which comprises an inhibitory endodomain, wherein the inhibitory endodomain comprises tyrosine kinase domain of C-terminal Src Kinase (CSK), which nucleic acid sequence has the following structure:
AgB2-spacer2-TM2-endo2
in which
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the inhibitory endodomain of the second CAR and
(iii) administering the cells from (ii) to the subject.

20. A method according to claim 18, wherein the disease is a cancer.

21-22. (canceled)


SEQUENCE LISTING (21 sequences)

SEQ ID NO: 1 - protein, 1085 aa, Artificial Sequence
  polypeptide comprising two chimeric antigen receptors (CAR)
MSLPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMAVPTQVLGLLLLWLTDARCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSMDPATTTKPVLRTPSPVHPTGTSQPQRPEDCRPRGSVKGTGLDFACDIYWAPLAGICVALLLSLIITLICYHRSRKRVCKLKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 2 - protein, 1278 aa, Artificial Sequence
  polypeptide comprising two CARs
MSLPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMAVPTQVLGLLLLWLTDARCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSMDPATTTKPVLRTPSPVHPTGTSQPQRPEDCRPRGSVKGTGLDFACDIYWAPLAGICVALLLSLIITLICYHRSRKRVCKSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 3 - protein, 21 aa, Artificial Sequence
  signal peptide
MGTSLLCWMALCLLGADHADG

SEQ ID NO: 4 - protein, 21 aa, Artificial Sequence
  signal peptide derived from IgG1
MSLPVTALLLPLALLLHAARP

SEQ ID NO: 5 - protein, 20 aa, Artificial Sequence
  signal peptide derived from CD8
MAVPTQVLGLLLLWLTDARC

SEQ ID NO: 6 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 7 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 8 - protein, 44 aa, Artificial Sequence
  spacer sequence, mouse CD8a stalk
ATTTKPVLRTPSPVHPTGTSQPQRPEDCRPRGSVKGTGLDFACD

SEQ ID NO: 9 - protein, 20 aa, Artificial Sequence
  spacer sequence, human IgG1 hinge
AEPKSPDKTHTCPPCPKDPK

SEQ ID NO: 10 - protein, 185 aa, Artificial Sequence
  spacer sequence, CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 11 - protein, 259 aa, Artificial Sequence
  spacer sequence, CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 12 - protein, 140 aa, Artificial Sequence
  sequence comprising CD28 transmembrane domain and CD3 Z endodomain
FWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 180 aa, Artificial Sequence
  sequence comprising CD28 transmembrane domain and CD28 and CD3 Zeta endodomains
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 216 aa, Artificial Sequence
  sequence comprising CD28 transmembrane domain and CD28, OX40 and CD3 Zeta endodomains
FWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 449 aa, Homo sapiens
  MISC_FEATURE: full length C-terminal Src kinase (CSK)
SAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 16 - protein, 256 aa, Artificial Sequence
  sequence of tyrosine kinase domain of CSK
LKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 17 - protein, 20 aa, Foot-and-mouth disease virus
RAEGRGSLLTCGDVEENPGP

SEQ ID NO: 18 - DNA, 3255 bp, Artificial Sequence
  dual CAR system using tyrosine kinase domain CSK as endodomain (CD19 and CD33)
atgagcctgcccgtgaccgccctgctgctgcccctggccctgctgctgcacgccgccagaccagacatccagatgacccagaccaccagcagcctgagcgccagcctgggcgaccgggtgaccatcagctgcagagccagccaggacatcagcaagtacctgaactggtaccagcagaagcccgacggcaccgtgaagctgctgatctaccacaccagccggctgcacagcggcgtgcccagccggttcagcggcagcggcagcggcaccgactacagcctgaccatcagcaacctggagcaggaggacatcgccacctacttctgccagcagggcaacaccctgccctacaccttcggaggcggcaccaagctggagatcaccaaggccggaggcggaggctctggcggaggcggctctggcggaggcggctctggcggaggcggcagcgaggtgaagctgcaggagtctggcccaggcctggtggccccaagccagagcctgagcgtgacctgcaccgtgagcggcgtgagcctgcccgactacggcgtgagctggatcaggcagcccccacggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactatggcggcagctacgctatggactactggggccagggcaccagcgtgaccgtgagctcagatcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatcttttgggtgctggtggtggttggtggagtcctggcttgctatagcttgctagtaacagtggcctttattattttctgggtgaggagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgcagagccgagggcaggggaagtcttctaacatgcggggacgtggaggaaaatcccgggcccatggccgtgcccactcaggtcctggggttgttgctactgtggcttacagatgccagatgtgacatccagatgacacagtctccatcttccctgtctgcatctgtcggagatcgcgtcaccatcacctgtcgagcaagtgaggacatttattttaatttagtgtggtatcagcagaaaccaggaaaggcccctaagctcctgatctatgatacaaatcgcttggcagatggggtcccatcacggttcagtggctctggatctggcacacagtatactctaaccataagtagcctgcaacccgaagatttcgcaacctattattgtcaacactataagaattatccgctcacgttcggtcaggggaccaagctggaaatcaaaagatctggtggcggagggtcaggaggcggaggcagcggaggcggtggctcgggaggcggaggctcgagatctgaggtgcagttggtggagtctgggggcggcttggtgcagcctggagggtccctgaggctctcctgtgcagcctcaggattcactctcagtaattatggcatgcactggatcaggcaggctccagggaagggtctggagtgggtctcgtctattagtcttaatggtggtagcacttactatcgagactccgtgaagggccgattcactatctccagggacaatgcaaaaagcaccctctaccttcaaatgaatagtctgagggccgaggacacggccgtctattactgtgcagcacaggacgcttatacgggaggttactttgattactggggccaaggaacgctggtcacagtctcgtctatggatcccgccaccacaaccaagcccgtgctgcggaccccaagccctgtgcaccctaccggcaccagccagcctcagagacccgaggactgccggcctcggggcagcgtgaagggcaccggcctggacttcgcctgcgacatctactgggcacctctggccggaatatgcgtggcactgctgctgagcctcatcatcaccctgatctgttatcaccgaagccgcaagcgggtgtgtaaactgaagctgctgcagaccatcggcaagggcgagtttggagatgtgatgctgggcgactaccggggcaacaaggtggcagtgaagtgcatcaagaacgacgctacagcccaggcttttctggccgaagccagcgtgatgacccagctgagacacagcaatctggtgcagctgctgggcgtgatcgtggaagaaaaaggcggcctgtatatcgtgaccgagtacatggccaagggcagcctggtggactacctgagaagtagaggcagaagcgtgctcggaggcgactgcctgctgaagtttagcctggatgtgtgcgaggctatggaatacctggaaggcaacaacttcgtgcaccgcgatctggccgccagaaatgtgctggtgtccgaggacaacgtggccaaggtgtccgatttcggcctgaccaaagaggccagcagcacccaggatacaggcaagctgcccgtgaaatggacagcccctgaggctctgagagagaagaagttcagcaccaagagcgacgtgtggtccttcggcatcctgctgtgggaaatctacagcttcggcagagtgccctatcctagaatccctctgaaggacgtggtgcccagagtggaaaagggctacaagatggatgcccctgacggctgtcctcctgccgtgtacgaagtgatgaagaactgctggcacctggacgccgctatgaggccatctttcctgcagctgagagagcagctggaacacatcaagacccacgagctgcacctg

SEQ ID NO: 19 - DNA, 3834 bp, Artificial Sequence
  dual CAR system using full sequence CSK as endodomain (CD19 and CD33)
atgagcctgcccgtgaccgccctgctgctgcccctggccctgctgctgcacgccgccagaccagacatccagatgacccagaccaccagcagcctgagcgccagcctgggcgaccgggtgaccatcagctgcagagccagccaggacatcagcaagtacctgaactggtaccagcagaagcccgacggcaccgtgaagctgctgatctaccacaccagccggctgcacagcggcgtgcccagccggttcagcggcagcggcagcggcaccgactacagcctgaccatcagcaacctggagcaggaggacatcgccacctacttctgccagcagggcaacaccctgccctacaccttcggaggcggcaccaagctggagatcaccaaggccggaggcggaggctctggcggaggcggctctggcggaggcggctctggcggaggcggcagcgaggtgaagctgcaggagtctggcccaggcctggtggccccaagccagagcctgagcgtgacctgcaccgtgagcggcgtgagcctgcccgactacggcgtgagctggatcaggcagcccccacggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactatggcggcagctacgctatggactactggggccagggcaccagcgtgaccgtgagctcagatcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatcttttgggtgctggtggtggttggtggagtcctggcttgctatagcttgctagtaacagtggcctttattattttctgggtgaggagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgcagagccgagggcaggggaagtcttctaacatgcggggacgtggaggaaaatcccgggcccatggccgtgcccactcaggtcctggggttgttgctactgtggcttacagatgccagatgtgacatccagatgacacagtctccatcttccctgtctgcatctgtcggagatcgcgtcaccatcacctgtcgagcaagtgaggacatttattttaatttagtgtggtatcagcagaaaccaggaaaggcccctaagctcctgatctatgatacaaatcgcttggcagatggggtcccatcacggttcagtggctctggatctggcacacagtatactctaaccataagtagcctgcaacccgaagatttcgcaacctattattgtcaacactataagaattatccgctcacgttcggtcaggggaccaagctggaaatcaaaagatctggtggcggagggtcaggaggcggaggcagcggaggcggtggctcgggaggcggaggctcgagatctgaggtgcagttggtggagtctgggggcggcttggtgcagcctggagggtccctgaggctctcctgtgcagcctcaggattcactctcagtaattatggcatgcactggatcaggcaggctccagggaagggtctggagtgggtctcgtctattagtcttaatggtggtagcacttactatcgagactccgtgaagggccgattcactatctccagggacaatgcaaaaagcaccctctaccttcaaatgaatagtctgagggccgaggacacggccgtctattactgtgcagcacaggacgcttatacgggaggttactttgattactggggccaaggaacgctggtcacagtctcgtctatggatcccgccaccacaaccaagcccgtgctgcggaccccaagccctgtgcaccctaccggcaccagccagcctcagagacccgaggactgccggcctcggggcagcgtgaagggcaccggcctggacttcgcctgcgacatctactgggcacctctggccggaatatgcgtggcactgctgctgagcctcatcatcaccctgatctgttatcaccgaagccgcaagcgggtgtgtaaaagcgccattcaggccgcttggccttctggcacagagtgtatcgccaagtacaacttccacggcaccgccgagcaggacctgcctttctgtaaaggcgacgtgctgaccatcgtggccgtgacaaaggaccccaactggtacaaggccaagaacaaagtgggcagagagggcatcatccccgccaactatgtgcagaagagagagggcgttaaggccggcaccaagctgtctctgatgccctggtttcacggcaagatcaccagagagcaggccgagagactgctgtaccctcctgaaaccggcctgttcctcgtgcgcgagagcacaaattaccccggcgactacaccctgtgtgtgtcctgtgatggcaaggtggaacactaccggatcatgtaccacgccagcaagctgagcatcgacgaggaagtgtacttcgagaacctgatgcagctggtcgagcactacacctccgatgccgatggcctgtgcaccagactgatcaagcccaaagtgatggaaggcaccgtggccgctcaggacgagttttacagatccggctgggctctgaacatgaaggaactgaagctgctgcagaccatcggcaagggcgagtttggagatgtgatgctgggcgactaccggggcaacaaggtggcagtgaagtgcatcaagaacgacgctacagcccaggcttttctggccgaagccagcgtgatgacccagctgagacacagcaatctggtgcagctgctgggcgtgatcgtggaagaaaaaggcggcctgtatatcgtgaccgagtacatggccaagggcagcctggtggactacctgagaagtagaggcagaagcgtgctcggaggcgactgcctgctgaagtttagcctggatgtgtgcgaggctatggaatacctggaaggcaacaacttcgtgcaccgcgatctggccgccagaaatgtgctggtgtccgaggacaacgtggccaaggtgtccgatttcggcctgaccaaagaggccagcagcacccaggatacaggcaagctgcccgtgaaatggacagcccctgaggctctgagagagaagaagttcagcaccaagagcgacgtgtggtccttcggcatcctgctgtgggaaatctacagcttcggcagagtgccctatcctagaatccctctgaaggacgtggtgcccagagtggaaaagggctacaagatggatgcccctgacggctgtcctcctgccgtgtacgaagtgatgaagaactgctggcacctggacgccgctatgaggccatctttcctgcagctgagagagcagctggaacacatcaagacccacgagctgcacctg

SEQ ID NO: 20 - protein, 967 aa, Artificial Sequence
  sequence of CAR construct comprising tyrosine kinase domain of CSK (tkCSK)
MSLPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMAVPTQVLGLLLLWLTDARCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSMDPATTTKPVLRTPSPVHPTGTSQPQRPEDCRPRGSVKGTGLDFACDIYWAPLAGICVALLLSLIITLICYHRSRKRVCKLKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSD

SEQ ID NO: 21 - protein, 4 aa, Artificial Sequence
  ITAM motif
  misc_feature (2)..(3): Xaa can be any naturally occurring amino acid
  MISC_FEATURE (4)..(4): Xaa may be Leu or Ile
YXXX